(19) United States
(12) Patent Application Publication
(10) Pub. No.: US 2023/0407247 A1
(43) Pub. Date: Dec. 21, 2023

(54) DEVICE AND METHOD FOR GROWING PLANT CELLS IN LIQUID CULTURE

(71) Applicant: Galy Co., Amherst, MA (US)

(72) Inventors: Luciano Luiz Bueno, Boston, MA (US); Shreyas Joshi, Amherst, MA (US); Franklin Magnum, Amherst, MA (US); David Dodds, Boston, MA (US)

(21) Appl. No.: 18/198,597

(22) Filed: May 17, 2023

Related U.S. Application Data
(60) Provisional application No. 63/342,904, filed on May 17, 2022.

(51) Int. Cl. C12N 5/04; C12P 19/04; C12M 1/24; C12M 1/00; C12M 3/00

(52) U.S. Cl. CPC C12N 5/04; C12P 19/04; C12M 23/48; C12M 23/14; C12M 23/08

(57) ABSTRACT

The present disclosure relates generally to the use of a plastic and/or pliable, sterile container, such as a bag, bottle, or pouch as a sterile enclosure for the culturing of plant cells in a liquid medium.

### TECHNICAL FIELD

The present disclosure relates generally to the use of a plastic and/or pliable, sterile container, such as a bag, bottle, or pouch as a sterile enclosure for the culturing of plant cells in a liquid medium.

### BACKGROUND

The use of biological methods, such as fermentation and cell culture for the production of pharmaceuticals is well known and widely practiced. The use of these methods for the production of commodity chemicals and materials is also well known, but such processes are much more sensitive to the cost of the process then pharmaceutical or other high-value products. A significant portion of the cost of any product made by such biological methods is the cost of the equipment that is required to execute the process.

Such equipment is generally installed in a facility in a permanent manner, and is termed “fixed equipment”. That is, as equipment that is permanently set in a specific place, or fixed, with a rigid support structure and connected to utilities, drains, and other services. Thus, it must remain in place during operation, cleaning, and sterilization. The interior spaces of these devices, which are usually referred to as bioreactors or fermentors, is the minimum area that must be rendered sterile, and in which the fermentation or cell culture is performed.

Generally, such equipment must be capable of being rendered sterile on all of its surfaces that will be in contact with the fermentation or cell culture. Moreover, once sterilized, the equipment must be physically capable of maintaining a sterile environment for the fermentation or cell culture throughout the entire growing process. As these processes may take days or weeks to complete, it is critical that the equipment be capable of maintaining a durable sterility over a long period of time. Thus, due to structural, thermal, and sterility concerns, bioreactors are generally made using stainless steel and/or glass components. As these materials are durable, expensive, and difficult to move, a pragmatic conservation of resources and simple economics dictate that, at the end of the process, the equipment can be largely cleaned, and subsequently reused for further cultures or fermentations. However, the down-time required to sterilize and configure a traditional, built-in bioreactor can severely hamper its throughput.

Additionally, often for the fermentation or cell culture to proceed, it must be continually and/or periodically supplied with nutrients, such as air, oxygen, carbohydrates, vitamins, nitrogenous materials, various inorganic materials, antibiotics, hormones and other growth factors, all while maintaining the sterile conditions of the process. Further, to track the progress of a culture of fermentation, it is preferred that samples may be removed periodically from an in-progress culture of fermentation, while also maintaining the sterile environment.

Moreover, bioreactors must often include a means to agitate, mix, blend or otherwise manipulate its contents, which is a requirement for many cell cultures or fermentations. Agitation/manipulation in a bioreactor may be used, for example, to physically move cells in the liquid media, to blend other solids entrained in the liquid media, to prevent the formation of stagnant areas or undesired concentration gradients in the liquid media, or to allow or enhance the dissolution of air or other gases into the liquid medium. To address this, mechanical agitators are often used, generally mounted to the top or the bottom of the fermentor or bioreactor. This requires further support structure, and thus expense.

Accordingly, creating a bioreactor that is capable of maintaining a durable sterility, allows for samples to be taken, allows for agitation, and provides a means for nutrient or other additions has traditionally required a large outlay in both space and expense. The conventional approach to increasing the amount of product that is made using a fermentation of cell culture process is to increase the size of the equipment, that is, the internal volume of the fermentor or bioreactor device is increased. This requires larger support structures, agitators, and piping to the bioreactor or fermentor, which further increases costs and upfront resources.

A fermentor or bioreactor device made using inexpensive materials that avoids the need for expensive vessels, which require sterilization and cleaning, attendant support structures, the requirement of a mechanical agitator, and is capable of growing plant cells in liquid culture, is therefore desirable. Further, the ability of such a device to be rapidly installed, easily moved, and deployed in large numbers in order to increase the amount product to be made by the process being practiced, is also desirable.

### SUMMARY

The present invention provides methods and systems that replace large, fixed bioreactors with bioreactors made using inexpensive materials, such as plastic, and even using pliable materials. These inexpensive materials are used to form sterile containers, such as bags, bottles, or pouches, which may be used as a sterile enclosure for the culturing of plant cells in a liquid medium. In particular, the presently disclosed systems and methods are used to manipulate (e.g., grow, feed, multiply, transform, transfect, expand, elongate, differentiate, etc.) plant cells using these sterile enclosures to produce products without the need to grow and cultivate mature plants. For example, in preferred aspects, these bioreactors are used to manipulate cotton cells to produce cotton fiber, without the need to grow cotton plants.

Cotton is the most widespread non-food crop in the world. However, cotton production is expensive both in terms of money and resources required for its successful cultivation. For example, cotton is a water-intensive crop, with an estimated 9,000-17,000 liters of water required for each kilogram of cotton fiber produced. This equates to enough drinking water to sustain 5,000 people for a day used in order to produce enough cotton to make two shirts.

The present Inventors discovered a process by which cotton cells may be cultured in a bioreactor to produce cotton fiber, without ever growing cotton plants. By using such methods of in vitro cotton production, cotton fiber can be produced using approximately 77% less water, 80% less land, and producing approximately 84% less carbon dioxide emissions than traditional in planta methods. Concurrently, the methods completely avoid the costs, transportation requirements, uncertainty and environmental dependency and impacts associated with traditional cotton agriculture.

However, in order to realize large-scale production of cotton fiber from cell cultures, the present Inventors sought a more economic, flexible, and rapidly deployable alternative to a series of large built-in, stainless-steel bioreactors. This led to the development of the methods and systems disclosed herein that use containers formed of inexpensive, plastic and/or pliable materials that fulfill the requirements of a “traditional” bioreactor to culture cotton cells.

The present invention is not limited to the manipulation of cotton cells to produce cotton fiber. The present invention may also be used in the production of other products from different types of plant cells, e.g., cacao cells, rice cells, cells from plants that produce desirable natural products such as the adjuvant QS21, paclitaxel, vanillin, latex, and flavors, fragrances, dyes, or other desirable substance that otherwise would require the cultivation of a plant to make a desirable product.

Accordingly, in certain aspects, the present invention provides a method for producing a plant product that includes providing a reaction vessel, wherein the reaction vessel has an average height and an average diameter, wherein a ratio between the height and diameter is termed the aspect ratio, such aspect ratio being between about 2 and 10; and in said reaction vessel, providing plant cells (e.g., cotton cells) and contacting said cells with a medium under conditions sufficient to manipulate the cells therein to reach a desirable physical state (e.g. a specific mass of cells) or physiological state (e.g. differentiation to a specific cell type, such as a cell capable of fiber production). A plant product, such as cotton fiber, is produced by the manipulated cells without the need for growing mature plants. This fiber may be harvested and used as any other cotton fiber.

In preferred aspects, the present invention provides a method for producing cotton fiber that includes providing a reaction vessel, wherein the reaction vessel has an average height and an average diameter, wherein the aspect ratio of the reaction vessel is between about 2 and 10; and in said reaction vessel, providing cotton cells and contacting said cells with a medium under conditions sufficient to manipulate at least a portion of said plurality of cotton cells. Preferably, in the case of cotton cells, manipulation includes one or more of growing, multiplying, differentiating, and/or elongating. Cotton fiber is produced by the manipulated cotton cells without the need for growing cotton plants. This fiber may be harvested and used as any other cotton fiber.

In certain aspects, methods of the invention further comprise aerating the mixture comprising plant cells and medium with compressed air or another gas or mixture of gases. Preferably, the mixture is aerated at a rate of at least 0.1 vvm. The aeration may agitate the mixture comprising plant cells without the need for a mechanical agitator. The aeration may be continuous, or pulsed intermittently, during multiplication or any other phase of the culture.

In certain aspects, the reaction vessel has a volume that is less than 100 gallons. The reaction vessel may have an aspect ratio of between about 2 and 10. Preferably, the aspect ratio about 3 and about 5.

In certain embodiments, the reaction vessel is a bottle that is less than 100 gallons. Preferably, the bottle is made from a thermoplastic, such as a polyethylene. The bottle may have a tapered end with a cap, and during the method the tapered end faces in a downward direction. The cap may comprises one or more through holes through which one or more of cells, gas, nutrients, and the medium or components thereof are provided to the bottle. The cotton cells may be collected from the tapered end of the bottle after a period of growth, or the cotton fiber may be collected after the cells have been appropriately manipulated.

In certain aspects, the method comprises providing a plurality of bottles as reaction vessels, and providing each bottle with the cells and medium (and optionally any other nutrient or medium, e.g., elongation medium) in parallel. The plurality of bottles may be independently provided with one or more of cells, gas, nutrients, and the medium from a central source.

In alternative embodiments, the reaction vessel is less than 100 gallons in volume and made using a pliable material, such as a plastic bag. The pliable reaction vessel may be placed into a rigid support before providing the cells and medium. The pliable reaction vessel may be provided with one or more of gas nutrients, and the cells and medium by one or more tubes, passing through holes punctured into the bag.

In certain aspects, the present invention provides systems for producing one or more plant products without the need to grow or cultivate mature plants. An exemplary system includes a reaction vessel, wherein the reaction vessel has an aspect ratio of between about 2 and 10; and in said reaction vessel, a mixture comprising plant cells. The system provides conditions in the reaction vessel to manipulate the plant cells. Once manipulated, the plant cells produce a plant product, without the need to ever grow or cultivate mature plants.

In preferred aspects, the present invention provides systems for producing cotton fiber from cotton cells, without the need for growing and cultivating cotton plants. An exemplary system includes a reaction vessel, wherein the reaction vessel has an aspect ratio of between about 2 and 10; and in said reaction vessel, a mixture comprising cotton cells. The system provides conditions in the reaction vessel to multiply the cotton cells. Once multiplied, the cotton cells may be further manipulated to produce cotton fiber, without the need to ever grow cotton plants. This cotton fiber may be collected and used in, for example, textile production.

In certain aspects, systems of the invention include one or more holes or ports that introduced compressed air or another gas or mixture of gasses into the reaction vessel to aerate the mixture of plant cells. Preferably, the system aerates the mixture at a rate of at least 0.1 vvm. The aeration may agitate the mixture comprising plant cells without the need for a mechanical agitator. The aeration may be continuous or intermittent during manipulation of the plant cells.

In certain aspects, the reaction vessel has a volume that is less than 100 gallons. The reaction vessel may have an aspect ratio of between about 2 and 10. Preferably, the ratio between the surface area and the volume is between about 3 and about 5.

In certain embodiments, reaction vessel is a bottle that is less than 100 gallons. Preferably, the bottle is made from a thermoplastic, such as a polyethylene. The bottle may have a tapered end with a cap, and during the method the tapered end faces in a downward direction. The cap may include one or more through holes through which one or more of cells, gas, nutrients, and the medium or components thereof are provided to the bottle. The plant product (e.g., cotton fiber) may be collected from the tapered end of the bottle.

In certain aspects, the system comprises a plurality of bottles as reaction vessels, and each bottle may include the solution of plant cells, allowing for cells to be manipulated amongst the reaction vessels in parallel. The system may provide the plurality of bottles with one or more of cells, gas, nutrients, and the medium from a central source.

In alternative embodiments of the systems disclosed herein, the reaction vessel is less than 100 gallons in volume and made using a pliable material, such as a plastic bag. The pliable reaction vessel may be placed into a rigid support before providing the solution. The pliable reaction vessel may be provided with one or more of cells, gas, nutrients, and the medium by one or more tube punctured into the bag.

### BRIEF DESCRIPTION OF THE FIGURES

FIG. 1 shows a pliable material formed into a bioreactor in accordance with the invention.

FIGS. 2A-2B show specific features that may be incorporated into a pliable bioreactor of the invention.

FIG. 3 shows a bottle used as a bioreactor in accordance with the invention.

### DETAILED DESCRIPTION OF THE INVENTION

The present invention provides methods and systems that replace large, fixed bioreactors with bioreactors made using inexpensive materials, such as plastic, and even using pliable materials. These inexpensive materials are used to form, sterile containers, such as bags, bottles, or pouches, which may be used as a sterile enclosure for the culturing of plant cells in a liquid medium. In particular, the presently disclosed systems and methods are used to produce plant products (e.g., cotton fiber) from cells cultured in the containers disclosed herein.

For example, in certain aspects, the pliable containers used in the methods and systems of the disclosure may include pouches or bags made from plastic. As a non-limiting example sealed plastic bags are provided as a roll of sterile plastic tubing, heat-sealed and perforated at a fixed length(s), such that an individual section of the roll can be detached from the roll of plastic tubing. A detached section of tubing forms a sealed, empty, plastic bag/pouch with a sterile environment in its interior. When this bag is fully filled with gas or liquid or a combination of both, it has an aspect ratio between 2 and 10. For example, a roll of sealed plastic tubing that is 1.57 feet in width will form a tube that is 3.14 feet in circumference when fully filled with gas or liquid, and have a diameter of 1.0 feet. Thus, if the roll of plastic tubing that is 1.57 feet in width is sealed and perforated at lengths from 2 to 10 feet, when a single detached section is completely filled with gas or liquid, it will give a filled plastic bag with an aspect ratio of 2 to 10. Accordingly, in certain aspects, preferred bioreactors used in the methods and systems of the invention include an aspect ratio of about 3 to about 5.

The size of the sections of the roll of plastic tubing may be of any width or length such that the aspect ratio of the fully filled section (the plastic bag) remains between 2 and 10, and the total volume of the plastic bag, when completely filled, does not exceed 100 US gallons in total volume.

While any suitable polymer for the plastic bag may be used, including opaque material that does not allow the admission of light into the interior of the plastic bag, it is convenient to use a polymer such as polyethylene, which is reasonably transparent and allows the contents of the bag to be directly observed.

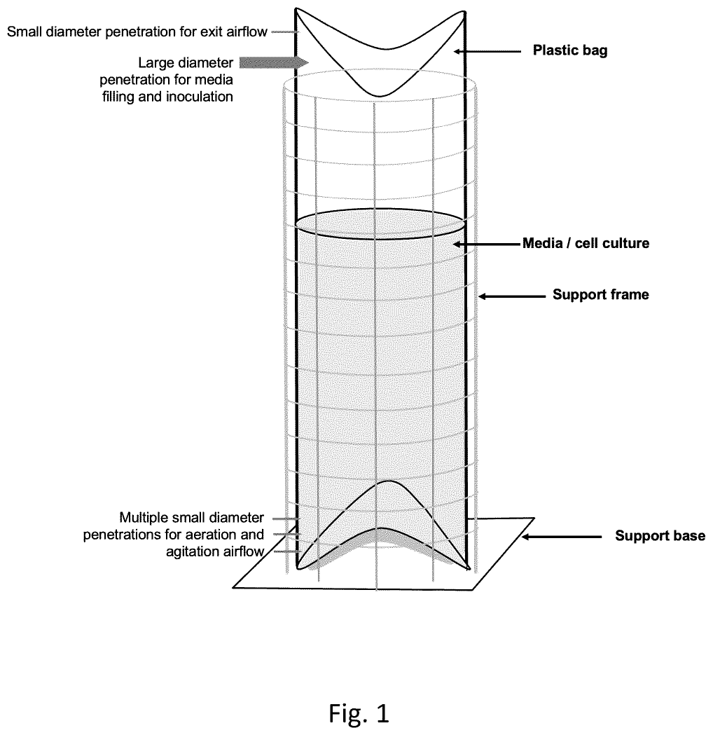

FIG. 1 provides a schematic of a pliable bioreactor of the invention made from a roll of plastic tubing. As shown, prior to filling, this empty, sealed plastic bag is loaded vertically into a cylindrical support structure made of any inexpensive material that will support the plastic bag in a vertical manner, having a base of suitable form that it will adequately support the bottom end of the plastic bag when it is filled.

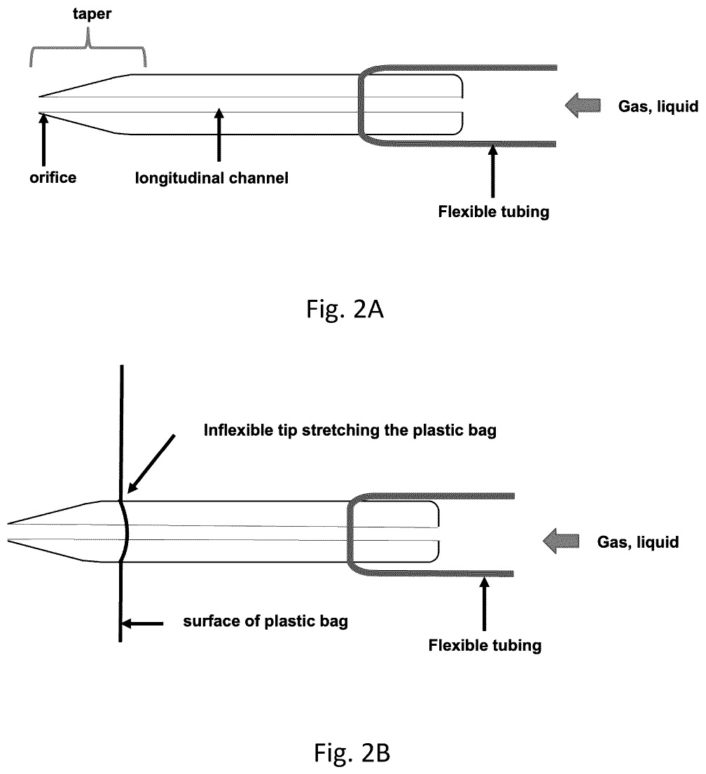

As shown in FIGS. 2A-2B, to admit gas or liquid into the empty, sealed plastic bag after it has been arranged in the vertical position on the base and inside the cylindrical supporting structure, an awl or similar tool is used to penetrate the plastic bag. Into the hole made by the awl or similar tool, a tip formed from a short piece of inflexible tubing is inserted. This inflexible tubing has a channel running longitudinally through its center capable of transmitting liquid or gas.

One end of the tip is tapered, so that it can be easily inserted into the hole formed by the awl or similar tool. The diameter of the non-tapered part of the tip is arranged such that it is slightly larger than the hole formed in the plastic bag by the awl or similar tool, and thus stretches the plastic slightly and forms a water-tight and gas-tight seal capable of withstanding the hydrostatic pressure caused by of a column of water filling the plastic tube.

The tip may be made of any inflexible made of material such as glass, metal, or plastic, that is capable of being sterilized in an autoclave, or by a similar method of sterilization, such as running steam through it. The non-tapered end of each tip is connected to flexible tubing through which gas or liquid may be delivered, passing through the tip and into the plastic tube, filling it with gas or liquid.

The orifice formed by the longitudinal channel at the tapered end of the inflexible tip may be of a size arranged to deliver a gas or liquid at a desired flow rate. More specifically, after the plastic tube has been filled with liquid media, the inflexible tip may be used to deliver air or other gas into the column of liquid media. In this specific use, the orifice of the tapered end of the inflexible tip may be of a diameter that produces a desired size of gas bubble in the liquid media.

Each inflexible tip is attached at the non-tapered end to flexible tubing that can be sterilized by autoclaving, steaming, or other methods while it is attached to the inflexible tip. Each piece of the flexible tubing with the inflexible tip at one end may be connected to a gas supply, such as compressed air or other gas, or to a source of water or liquid media. A sterilizing filter may be placed in the flexible tubing prior to the inflexible tip, such that any gas or liquid traveling through the flexible tubing is sterilized by filtration prior to passing through the inflexible tip and into the plastic bag. A clamp or other control device may be used on the flexible tubing to control the flow of gas or liquid through it and trough the inflexible tip.

In operation, at least two inflexible tips are inserted into the individual plastic bag, using the awl or similar tool to make the initial penetration of the plastic bag.

Multiple inflexible tips may be inserted into the plastic bag situated in the vertical position inside the support structure. Multiple diameters of inflexible tips may be inserted into the bag, first perforating the plastic bag with an awl or similar tool of a suitable diameter such that the inflexible tip is inserted it will stretch the plastic bag sufficiently to form a seal capable of withstanding the pressure of gas or liquid inside the filled plastic bag.

Conversely, an inflexible tip previously inserted in the plastic bag can be removed, and the remaining hole covered with a piece of tape, thus preventing air or liquid form escaping. Typically, this is only done with penetrations that are not exposed to the liquid contents of the plastic bag, that is, are above the level of any liquid inside the plastic bag.

By admitting a sufficient gas flow thorough a sufficient number of inflexible tips located near the bottom of the plastic bag, any liquid, solids such as cell mass, inside the plastic bag, may be agitated without the need of a mechanical agitator. The flexible tubing providing air or other gas to the inflexible tips may be metered or controlled in any convenient manner that allows the desired levels of aeration and agitation of the contents of the plastic bag. Inflexible tips may be inserted at the top of the plastic bag to operate as exit ports for exhaust air and off-gasses. The flexible tubing connected to these inflexible tips may be run to a gas analyzer, or a flow controller to allow the plastic bag to remain appropriately inflated.

It is considered desirable to insert a sufficient number of inflexible tips, with sufficiently large orifices, and attached to a sufficiently large compressed air or other gas supply, to allow a rate of aeration of at least 0.1 vvm or higher.

Sensors can also be inserted into the plastic bag, if appropriately sterilized and an awl of similar tool of appropriate diameter is used. For example, pH or dissolved oxygen sensors.

When penetrating the plastic bag with the awl or similar tool, and the insertion of the tapered inflexible tip into the plastic bag, sterile technique must be followed to ensure that the interior of the plastic bag and whatever contents there may be inside it, are not contaminated. This can be achieved by spraying the surface of the plastic bag and the awl ore similar tool with a disinfecting solution, such as ethanol or isopropanol. Following perforation of the plastic bag, disinfecting solution is again applied to the local surface of the plastic bag and the inflexible tip.

In one embodiment, the device is used in the following manner:
a. A section of the roll of sealed, empty sterile plastic tubing is removed to give an individual empty, sealed, sterile plastic bag.
b. The empty, sealed, sterile plastic bag is loaded into the cylindrical support structure, such that the sealed plastic bag is in a vertical position and touching the supporting base.
c. The plastic bag is perforated using the awl or similar tool, and an inflexible tip attached to a length of flexible tubing connected to a compressed air supply and having a sterilizing filter in-line such that air reaching the interior plastic bag is filter-sterilized.
d. The bag is allowed to inflate until it fills the support structure and rests fully on the base at the bottom of the support structure.
e. The inflated plastic bag may perforated multiple times, to allow the insertion of another inflexible tip with flexible tubing to allow the introduced air to exit from the plastic bag. The flexible tubing through which air is exiting the bag may be clamped or otherwise manipulated to control the level of inflation of the plastic bag.
f. Additional inflexible tips are inserted into the inflated plastic bag in order to deliver sterile media. Usually, this is done with a perforation at the top of the plastic bag, but it can be at any convenient location. The diameter of the inflexible tip can be as large as necessary to allow the introduction of liquid media in a convenient time, provided the diameter is not so large as to compromise the integrity of the plastic bag.
g. Similarly, a volume of inoculum containing growing cells, may be introduced through an inflexible tip of appropriate diameter penetrating the plastic bag. In this case, no sterilizing filter is placed in the flexible tubing, and the entire length of flexible tubing attached to the inflexible tip must be previously sterilized.
h. In a similar manner, additional inflexible tips may be inserted at the bottom of the bag, attached to flexible tubing with sterilizing filters and connected to a gas supply, such as compressed air, to allow aeration of the media, and later, agitation of cell mass.
i. Any inflexible tips that are no longer considered necessary may be removed, and the remaining hole sealed with a piece of adhesive tape.

The plastic bag is now filled with sterile liquid media, the inoculum of the desired cells, and is appropriately aerated and agitated with a metered or controlled flow of air of other gas. Inflexible tips inserted at the top of the plastic bag operate as exit ports, and gas flow through them may be controlled or monitored as desired.

The entire device may be located in an environment of the desired temperature, such as incubator, warm-room, or other area with controlled temperature. No active temperature of the plastic bag itself is required.

At the end of the desired period of growth in the plastic bag, the contents may be harvested or recovered by inserting an inflexible tip with attached flexible tubing at the bottom of the plastic bag, and allowing the contents of the plastic bag to exit via gravity, or to be pumped out.

After the bag is emptied, the inflexible tips are all removed for future use, and the empty bag may be discarded in any appropriate manner.

The plastic bag may be made of a polymer that can be recycled, or has been produced from a sustainable source, e.g., polyethylene produced from the chemical manipulation of ethanol, such ethanol having been produced by fermentation of a sustainable carbohydrate source, such as corn starch or sugarcane.

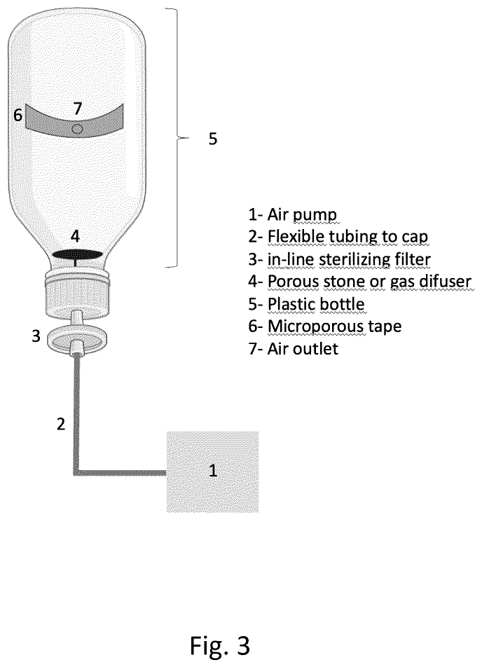

Alternatively, the methods and systems of the invention may use inexpensive bottles as bioreactors for producing plant products (e.g., cotton fiber) from plant cells, without growing or cultivating plants. For example, the bottle may be a plastic bottle. FIG. 3 provides a schematic of an exemplary bottle bioreactor of the invention. As shown, the bottle has openings closed with a threaded cap. As known in the art bottles with of different sizes are available in many different polymers with many different cap diameters and threads. The caps are also available in a variety of polymers.

While any suitable polymer for the plastic bottle may be used, including opaque material that does not allow the admission of light into the interior of the plastic bottle, it is convenient to use a polymer such as polyethylene, which is reasonably transparent and allows the contents of the bottle to be directly observed.

The plastic bottle may be of any convenient size and shape, such that it does not exceed a total volume of 100 US gallons, and has an aspect ratio in the range of 2 to 10. It is desirable to have the top of the bottle tapered from the diameter of the main body of the bottle to the threaded opening, and this taper may be presented as rounded shoulders rather than a true conical taper. The length and degree of this taper, or geometry of the shoulders, will be chosen with respect to the physical behavior and size of the cells to be grown in the bottle bioreactor.

The plastic bottle is mounted in a support structure such that the threaded opening and cap are at the bottom. Gases and liquids that are to be admitted into the bottle bioreactor will be added through the threaded opening via a modified cap.

The bottle itself requires only the addition of one or more holes in the bottom of the bottle. In the inverted position in which the bottle will be used, these holes will be at the top of the bioreactor, and will be used as exit ports for exit gases. The holes should be of a suitable diameter to allow any exit air or off-gases to escape without building up undue pressure in the bottle bioreactor. To prevent contamination, these holes may be covered with a porous tape, of the kind used to cover bandages and wounds, or “breathable” tape used to seal outdoor garments or rain gear.

The cap requires more modification. One or more holes are created in the cap to allow gases or liquids to be admitted into the bottle bioreactor. A single hole in the cap may serve as an inlet for both gases and liquids, but it is preferable to have one or more holes of smaller diameter for gas admission, with at least one hole of larger diameter for admission of liquid media and inoculum. In this manner the cap is permanently modified, and hose-barb nipples may be glued or otherwise attached to the cap at each added hole. Flexible tubing may then be run onto these hose barbs. If the cap is very large, and the holes are also large, then different fittings for flexible tubing may be used instead of hose barbs, for example, pressure connectors, threaded connectors and the like.

The support structure holding the bottle in the inverted position may be of any suitable material and size, such that the threaded opening of the bottle or the cap does not bear any of the support load, the cap is accessible, and any tubing running to holes in the cap is not kinked. The bottle does not have to be rigidly held in the support structure, and it is preferable that the load carried on the support structure is passed from the shoulders or taper of the plastic bottle. In this manner, when the plastic bottle bioreactor is empty, the cap may be unscrewed form the threaded opening, and the bottle simply lifted out of the support structure.

In preferred aspects, the bottle and cap may both be re-used multiple times.

In one embodiment, the device is used in the following manner:
a. A plastic bottle is obtained, and modified by the addition of at least one hole in or near the bottom of the bottle. The hole is sealed with microporous tape.
b. The cap of the bottle is modified by the addition of at least one hole, and a suitable connector for attaching flexible tubing to the cap is added to the cap.
c. To allow gas admitted through the cap to form bubbles of a desired size, a porous stone or diffuser may be attached to the cap on the inside of one of the inlet holes.
d. The bottle is placed in an inverted position in the support structure, and the cap screwed into place.
e. A sterilizing solution is admitted through all of the tubing that is attached to the holes in the cap, until the plastic bottle is completely filled. The solution is allowed to remain for sufficient time to sanitize the inside of the plastic bottle, the connections through the cap, and the flexible tubing running to the cap. After this time has passed, the sanitizing solution is drained out of the plastic bottle bioreactor through the flexible tubing. In this manner the interior of the plastic bottle bioreactor and the associated tubing are sanitized.
f. After sanitization, inline sterilizing filters may be fitted to the flexible tubing running to the cap. Any inoculum to be admitted to the plastic bottle bioreactor will require a line of flexible tubing that does not have an inline filter.
g. A volume of media is then pumped into the plastic bottle bioreactor through the flexible tubing attached to the cap, to the desired fill volume.
h. The desired amount of inoculum is then added through flexible tubing attached to the cap, provided that it does not pass through a sterilizing filter.
i. After the plastic bottle bioreactor is charges with media and is inoculated, air or other gases may be admitted through the cap, and through the optional diffuser, at the desired rate.

The entire device may be located in an environment of the desired temperature, such as incubator, warm-room, or other area with controlled temperature. No active temperature of the plastic bottle itself is required.

At the end of the desired period of growth in the plastic bottle, the contents may be harvested or recovered through the flexible tubing in the cap.

After the plastic bottle is emptied, the cap may be unscrewed, and the plastic bottle removed from the support structure for cleaning and re-use.

The plastic bottle may be made of polymers that can be recycled, or has been produced from a sustainable source, e.g., polyethylene furanoate or polyethylene terephthalate, such polymers having been produced from renewable feedstock, such as glucose, sucrose, cellulose or hemicellulose.

It will be clear that a plurality of plastic bottle bioreactors may be attached together in a parallel manner, with all liquids and gases to be admitted being supplied through common plenum or header assemblies. In this manner, increased scale is achieved in a modular manner simply by adding a desired number of bioreactors.

1. A method for producing a plant product from plant cells, comprising steps of:
(a) providing a reaction vessel, wherein the reaction vessel has an average height and an average diameter, wherein a ratio between the height and diameter is between about 2 and 10; and
(b) in the reaction vessel, providing plant cells and contacting the plant cells with a liquid medium under conditions sufficient to induce at least a plurality of the plant cells to be manipulated to yield a plurality of manipulated plant cells, thereby producing the plant product.

2. The method of claim 1, wherein during the step (b) the plant cells are manipulated by one or more of growing the cells, expanding the cells, maturing the cells, elongating the cells, transfecting the cells, or differentiating the cells.

3. The method of claim 1, wherein during the step (b) the liquid medium contacting the plant cells is aerated with compressed air or another gas or mixture of gasses.

4. The method of claim 3, wherein the liquid medium is aerated at a rate of at least 0.1 vvm.

5. The method of claim 4, wherein the aeration agitates the liquid medium contacting the plant cells without need for a mechanical agitator.

6. The method of claim 4, wherein the aeration is continuous while the plant cells are being manipulated.

7. The method claim 4, wherein the aeration is intermittent while the plant cells are being manipulated.

8. The method of claim 1, wherein the reaction vessel has a volume that is less than 100 US gallons.

9. The method of claim 8, wherein the reaction vessel has an aspect ratio between about 2 and 10.

10. (canceled)

11. The method of claim 1, wherein the reaction vessel is a bottle that is less than 100 US gallons.

12-14. (canceled)

15. The method of claim 11, wherein the bottle has a tapered end with a cap, and during the step (b) the tapered end faces in a downward direction.

16. The method of claim 15, wherein the cap comprises one or more through holes through which one or more of gas, nutrients, or the liquid medium or components thereof are provided to the bottle.

17. The method of claim 15, wherein the plant product is collected from the tapered end of the bottle.

18-19. (canceled)

20. The method of claim 1, wherein the reaction vessel is less than 100 US gallons in volume and made using a pliable material.

21. The method of claim 20, wherein the reaction vessel is a plastic bag.

22. (canceled)

23. The method of claim 20, wherein the reaction vessel is provided with one or more of cells, gas, nutrients, or the liquid medium by one or more tubes punctured into the bag by means of an inflexible tip.

24. (canceled)

25. The method of claim 1, wherein the plant product is cotton fiber and the plant cells are cotton cells.

26. The method of claim 25, wherein during the step (b) the cotton cells are manipulated by elongating the cotton cells.

27. The method of claim 25, wherein during the step (b) the cotton cells are manipulated by propagating the cotton cells.

28. A system for producing a plant product from plant cells, comprising:
(a) a reaction vessel, wherein the reaction vessel has an average height and an average diameter, wherein a ratio between the height and diameter is between about 2 and 10; and
(b) in the reaction vessel, plant cells and a liquid medium,
wherein the plant cells and the liquid medium are under conditions sufficient to induce at least a plurality of the plant cells to be manipulated to produce the plant product.

29-52. (canceled)
